(19) United States
(12) Patent Application Publication
(10) Pub. No.: US 2025/0366715 A1
(43) Pub. Date: Dec. 4, 2025

(54) CALIBRATION METHOD FOR MEDICAL IMAGING SYSTEM, AND MEDICAL IMAGING SYSTEM

(71) Applicant: GE Precision Healthcare LLC, Waukesha, WI (US)

(72) Inventors: Qingyu Dai, Beijing (CN); Xiaolan Liu, Beijing (CN); Changbo Liu, Beijing (CN); Qilin Lu, Beijing (CN); Kun Wang, Beijing (CN)

(21) Appl. No.: 19/221,124

(22) Filed: May 28, 2025

(30) Foreign Application Priority Data
May 29, 2024 (CN) 202410677319.1

(51) Int. Cl. A61B 5/00

(52) U.S. Cl. CPC A61B 5/0033; A61B 2560/0223

(57) ABSTRACT

Aa calibration method for a medical imaging system and a medical imaging system includes: obtaining a first image including a reference object and a scanned subject, and determining a first distance according to the first image. The first distance includes a distance from at least one point of the scanned subject to the reference object. The method further includes moving the scanned subject to a preset position according to the first distance and a second distance from the reference object to the preset position; and calibrating a parameter of the medical imaging system by using the scanned subject at the preset position.

### CROSS REFERENCE TO RELATED APPLICATIONS

The present application claims priority and benefit of Chinese Patent Application No. 202410677319.1 filed on May 29, 2024, which is incorporated herein by reference in its entirety.

### TECHNICAL FIELD

Embodiments of the present application relate to the technical field of medical devices, and in particular, to a calibration method for a medical imaging system, and a medical imaging system.

### BACKGROUND

A medical imaging system is generally used to perform medical imaging on a detected subject (for example, a patient) to obtain internal physiological information of the detected subject. For example, the medical imaging system may be used to obtain images of a patient's skeletal structure, brain, heart, lungs, and various other features.

Before the medical imaging is performed on the detected subject by using the medical imaging system, the medical imaging system needs to be calibrated. For example, the medical imaging system is used to perform pre-scanning on a scanned subject, and relevant parameters of the medical imaging system are calibrated according to a pre-scanning result and pre-stored information of the scanned subject, so as to ensure the reliability of the medical imaging system.

### SUMMARY

The inventor has found that in the process of calibrating a medical imaging system, a scanned subject needs to be positioned so as to move the scanned subject to a preset position, thereby performing calibration by using the scanned subject at the preset position.

Currently, a laser apparatus is used to position the scanned subject. For example, after the scanned subject is placed with reference to a position of laser irradiated by the laser apparatus, the scanned subject is moved to the preset position.

In the foregoing manner, the laser apparatus needs to be provided in the medical imaging system, which leads to high costs of the medical imaging system. In addition, the foregoing manner requires that a placement position of the scanned subject is strictly aligned with an irradiation position of the laser. If an operator does not place the scanned subject at the position aligned with the irradiation position of the laser, the scanned subject cannot be reliably moved to the preset position. As a result, the accuracy of a calibration result of the medical imaging system cannot be ensured. In addition, the use of the laser introduces safety concerns.

For at least one of the foregoing problems, embodiments of the present application provide a calibration method for a medical imaging system, and a medical imaging system.

According to one aspect of the embodiments of the present application, a calibration method for a medical imaging system is provided. The method comprises: obtaining a first image comprising a reference object and a scanned subject, and determining a first distance according to the first image, wherein the first distance comprises a distance from at least one point of the scanned subject to the reference object; moving the scanned subject to a preset position according to the first distance and a second distance from the reference object to the preset position; and calibrating a parameter of the medical imaging system by using the scanned subject at the preset position.

According to one aspect of the embodiments of the present application, a medical imaging system is provided. The system comprises: a controller, configured to perform the calibration method for a medical imaging system described above; and an examination table, configured to place a scanned subject and move the scanned subject to a preset position.

One of the beneficial effects of the embodiments of the present application is that: the first image comprising the reference object and the scanned subject is obtained, and the first distance from the at least one point of the scanned subject to the reference object is determined according to the first image; the scanned subject is moved to the preset position according to the first distance and the second distance from the reference object to the preset position; and a parameter of the medical imaging system is calibrated by using the scanned subject at the preset position. In this way, the accuracy of the calibration result of the medical imaging system can be improved, which is beneficial to simplify the calibration process and reduce the costs of the medical imaging system.

With reference to the following description and drawings, specific implementations of the embodiments of the present application are disclosed in detail, and the way in which the principles of the embodiments of the present application can be employed are illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a magnetic resonance imaging system according to an embodiment of the present application;

FIG. 2 is a schematic diagram of a calibration method for a medical imaging system according to an embodiment of the present application;

FIG. 3 is a schematic diagram of the implementation of step 201 according to an embodiment of the present application;

FIG. 4 is a schematic diagram of the implementation of step 302 according to an embodiment of the present application;

FIG. 5 is a schematic diagram of a first height, a second height, a fourth distance, an offset, and a third distance according to an embodiment of the present application;

FIG. 6 is a schematic diagram of a partial structure of a medical imaging system according to an embodiment of the present application;

FIG. 7 is another schematic diagram of a calibration method for a medical imaging system according to an embodiment of the present application;

FIG. 8 is a schematic diagram of a scanned subject, a reference object, and an examination table;

FIG. 9 is another schematic diagram of a scanned subject, a reference object, and an examination table;

FIG. 10 is another schematic diagram of a calibration method for a medical imaging system according to an embodiment of the present application;

FIG. 11 is a schematic diagram of a marker, a reference object, and an examination table according to an embodiment of the present application;

FIG. 12 is a schematic diagram of a calibration apparatus for a medical imaging system according to an embodiment of the present application; and

FIG. 13 is another schematic diagram of a calibration apparatus for a medical imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the” include the plural forms, and should be broadly construed as “a type of” or “a class of” rather than being limited to the meaning of “one”. In addition, the term “the” should be construed as including both the singular and plural forms, unless otherwise specified in the context. In addition, the term “according to” should be construed as “at least in part according to . . . ”, and the term “based on” should be construed as “at least in part based on . . . ”, unless otherwise specified in the context.

In the embodiments of the present application, the term “scanned subject” may be equivalently replaced with “subject”, “subject to be scanned”, “subject being scanned”, “patient”, “subject of study”, or the like, and the “scanned subject” may be a living being such as a human being or an animal, or an inanimate object.

In the embodiments of the present application, the term “include/comprise” when used herein refers to the presence of features, integrated components, steps, or assemblies, but does not preclude the presence or addition of one or more other features, integrated components, steps, or assemblies.

The features described and/or illustrated for one implementation may be used in one or more other implementations in the same or similar way, be combined with features in other embodiments, or replace features in other implementations.

In the embodiments of the present application, a medical imaging system is applicable to a variety of medical imaging scenarios, including, but not limited to, magnetic resonance imaging (MRI), computed tomography (CT), positron emission computed tomography (PET), single photon emission computed tomography (SPECT), PET/CT, PET/MR, or any other suitable medical imaging scenarios.

In the embodiments of the present application, the method, apparatus and system of the present application are exemplarily described by taking an MRI scenario as an example. It should be understood that the contents of the embodiments of the present application are also applicable to other medical imaging scenarios.

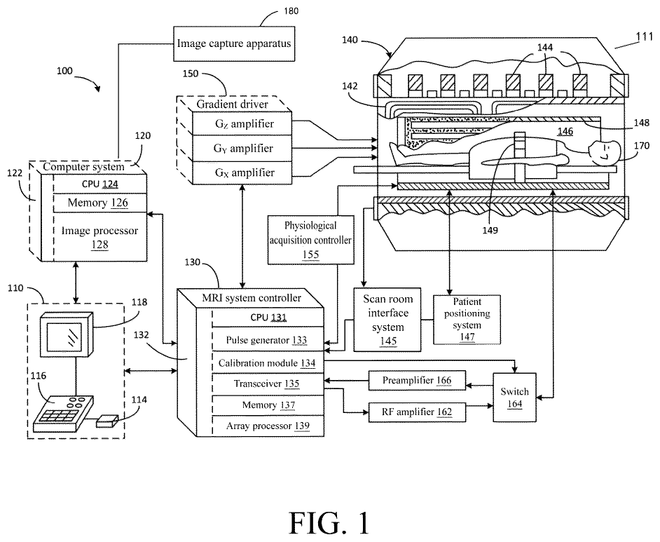

FIG. 1 is a schematic diagram of a magnetic resonance imaging (MRI) system 100 according to an embodiment of the present application.

As shown in FIG. 1, the MRI system 100 includes a scanning unit 111. The scanning unit 111 is used to perform a magnetic resonance scan of a subject (e.g., a human body) 170 to generate image data of a region of interest of the subject 170, wherein the region of interest may be a pre-determined anatomical site or anatomical tissue.

The operation of the MRI system 100 is controlled by an operator workstation 110 that includes an input device 114, a control panel 116, and a display 118. The input device 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and in communication with a computer system 120 that enables an operator to control the generation and display of images on the display 118. The computer system 120 includes various components that communicate with one another by means of an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by medical imaging functions implemented in the CPU 124. The computer system 120 may be connected to an archive media device, a persistent or backup memory, or a network. The computer system 120 may be coupled to and communicates with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator (also known as pulse generator) 133 in communication with the operator workstation 110, a calibration module 134 configured to calibrate a medical imaging system, a transceiver (also known as RF transceiver) 135, a memory 137, and an array processor 139.

In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be performed during an MRI scan, so as to be used to control the scanning unit 111 to perform the flow of the aforementioned magnetic resonance scan. The MRI system controller 130 is further coupled to a gradient driver system (also known as gradient driver) 150 and is in communication therewith, and the gradient driver system is coupled to a gradient coil assembly 142 to generate a magnetic field gradient during an MRI scan.

The sequence pulse generator 133 may further receive data from a physiological acquisition controller 155 that receives signals from a plurality of different sensors (e.g., electrocardiogram (ECG) signals from electrodes attached to a patient, etc.), the sensors being connected to a subject or patient 170 undergoing an MRI scan. The sequence pulse generator 133 is coupled to and in communication with a scan room interface system 145 that receives signals from various sensors associated with the state of the resonance assembly 140. The scan room interface system 145 is further coupled to a patient positioning system 147 and is in communication therewith, and the patient positioning system 147 sends and receives signals to control a patient table (e.g., an examination table) to move to a desired position for an MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field B₀ throughout a cylindrical imaging volume 146. The resonance assembly 140 further includes an RF body coil 148, which, in operation, provides a transverse magnetic field B₁, the transverse magnetic field B₁ being substantially perpendicular to B₀ throughout the entire cylindrical imaging volume 146. The resonance assembly 140 may further include an RF surface coil 149 for imaging different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in the k-space, the y direction may be referred to as a phase encoding direction or a k_{y} direction in the k-space, and the z direction may be referred to as a layer surface selection (layer selection) direction. Gₓ can be used for frequency encoding or signal readout, and is generally referred to as a frequency encoding gradient or a readout gradient. G_{y} can be used for phase encoding, and is generally referred to as a phase encoding gradient. G_{z} can be used for slice (layer) position selection to obtain k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses amplified by an RF amplifier 162, and provides the same to the RF body coil 148 through a transmit/receive switch (also known as T/R switch or switch) 164.

As described above, the RF body coil 148 and the RF surface coil 149 may be used to transmit RF excitation pulses and/or receive resulting MR signals from the patient undergoing the MRI scan. The MR signals emitted by excited nuclei in the patient of the MRI scan may be sensed and received by the RF body coil 148 or the RF surface coil 149 and sent back to a preamplifier 166 through the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be obtained by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image to be reconstructed, the data is rearranged into separate k-space data arrays, and each of said separate k-space data arrays is input to the array processor 139, the array processor being operated to transform the data into an array of image data by Fourier transform.

The array processor 139 uses transform methods, most commonly Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program used to implement scanning processing (such as a scan flow and an imaging sequence), image reconstruction, medical imaging, etc. For example, the storage medium may store a program used to implement the magnetic resonance imaging method according to the embodiments of the present invention. The described storage medium may include, for example, a ROM, a floppy disk, a hard disk, an optical disk, a magneto-optical disk, a CD-ROM, or a non-volatile memory card.

The MRI system 100 further includes an image capture apparatus 180 (also known as photographing unit). The image capture apparatus 180 is configured to obtain information such as information of other components of the medical imaging system and/or visual and morphological information of a scanned subject. In general, the image capture apparatus 180 may be installed near an examination table so that image information of a component such as the examination table and/or the scanned subject can be maximally collected in a non-contact manner. The image information may be used to assist with medical imaging operations. For example, the image information obtain by the image capture apparatus 180 may be used for keypoint recognition of the subject (that is, landmark recognition), so that a subsequent scanning operation may be performed according to a result of the keypoint recognition. The present application is not limited thereto. The image information may also be used to other operations.

Description is made below in conjunction with the embodiments.

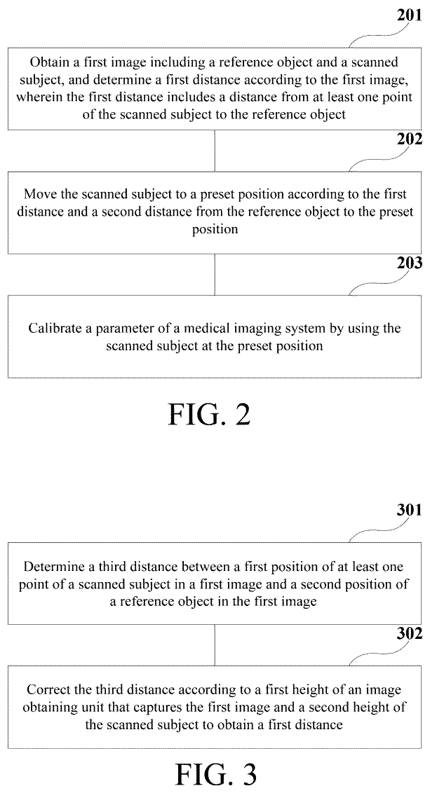

An embodiment of the present application provides a calibration method for a medical imaging system. FIG. 2 is a schematic diagram of a calibration method for a medical imaging system according to an embodiment of the present application. As shown in FIG. 2, the method includes: at step 201: obtaining a first image including a reference object and a scanned subject, and determining a first distance according to the first image, the first distance including a distance from at least one point of the scanned subject to the reference object. The method also includes at step 202: moving the scanned subject to a preset position according to the first distance and a second distance from the reference object to the preset position; and at step 203: calibrating a parameter of the medical imaging system by using the scanned subject at the preset position.

According to the foregoing embodiment, the first image including the reference object and the scanned subject is obtained, and the first distance from the at least one point of the scanned subject to the reference object is determined according to the first image; the scanned subject is moved to the preset position according to the first distance and the second distance from the reference object to the preset position; and the parameter of the medical imaging system is calibrated by using the scanned subject at the preset position. In this way, the accuracy of a calibration result of the medical imaging system can be improved, which is beneficial to simplify the calibration process and reduce the costs of the medical imaging system.

Specifically, in the foregoing method, the relative position of the scanned subject and the reference object is determined according to the first image including the reference object and the scanned subject. In this way, there is no need to set a laser apparatus in the medical imaging system, so that the costs of the medical imaging system can be reduced, and the safety problem caused by the use of laser can be avoided. Because the actual placement position of the scanned subject can be determined according to the first image, in the calibration process, the operator only needs to place the scanned subject within an approximate position range, and does not need to repeatedly adjust the position of the scanned subject. In this way, the calibration process can be simplified, and the accuracy of the calibration result of the medical imaging system can be improved.

In some embodiments, in step 201, the first image may be obtained in various manners. For example, the first image including the reference object and the scanned subject may be captured/generated by an image capture apparatus (a camera, a video camera, or the like). Alternatively, the first image stored in advance may be obtained from a storage apparatus.

In some embodiments, in step 201, the first distance from the at least one point of the scanned subject to the reference object may be determined according to the first image.

The at least one point of the scanned subject may be any one or more points on the scanned subject. For example, the at least one point of the scanned subject may include a center point of the scanned subject. A distance from the center point of the scanned subject to the reference object is determined, so that the center point of the scanned subject can be located at the preset position when the scanned subject is moved. In this way, the calibration operation is simplified.

In some embodiments, the first distance from the at least one point of the scanned subject to the reference object may be a distance in a first direction in which the scanned subject is moved in step 202. The present application is not limited thereto. The first distance may alternatively be a distance in another direction, and in this case, the distance in the first direction may be determined according to the first distance. Similarly, the second distance in step 202 may alternatively be the distance in the first direction, or a distance in another direction.

In some embodiments, in step 201, the first distance may be determined in various manners.

For example, a first position of the at least one point of the scanned subject in the first image and a second position of the reference object in the first image may be determined, and a distance (that is, a third distance) between the first position and the second position may be used as the first distance.

For another example, because height planes of objects in the first image may be different, there is a position offset due to the angle of view. To ensure the accuracy of the first distance, the third distance may be corrected, and a corrected third distance is used as the first distance.

FIG. 3 is a schematic diagram of the implementation of step 201 according to an embodiment of the present application. As shown in FIG. 3, the method for determining the first distance in step 201 may include: at step 301: determining a third distance between a first position of the at least one point of the scanned subject in the first image and a second position of the reference object in the first image. Further the method includes at step 302: correcting the third distance according to a first height of a photographing unit that captures the first image and a second height of the scanned subject to obtain the first distance.

Therefore, the first distance can be accurately determined, so that the scanned subject can be reliably moved to the preset position, thereby ensuring the reliability of a calibration result.

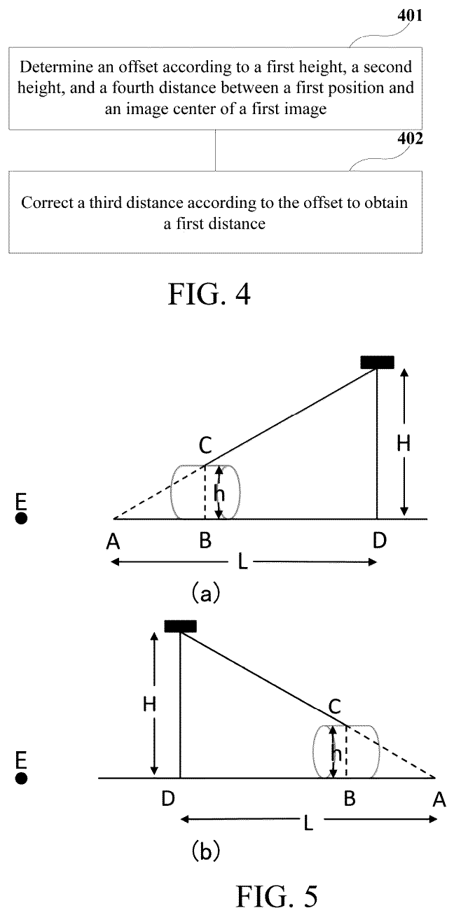

In some embodiments, in step 302, the third distance may be corrected in various manners. FIG. 4 is a schematic diagram of the implementation of step 302 according to an embodiment of the present application. As shown in FIG. 4, step 302 may include: a) Step 401: determining an offset according to the first height, the second height, and a fourth distance between the first position and an image center of the first image; and b) Step 402: correcting the third distance according to the offset to obtain the first distance.

In some embodiments, the first height of the photographing unit and the second height of the scanned subject may be heights relative to a same reference plane. In this way, the accuracy of the offset can be ensured.

In some embodiments, the reference plane may be at the same height as the reference object, or the height difference between the reference plane and the reference object is less than a preset value. In this way, the accuracy of the first distance can be further ensured.

In some embodiments, the first height of the photographing unit may be determined by depth information of the first image. For example, the depth of the reference plane in the first image may be used as the first height of the photographing unit. The present application is not limited thereto. The first height of the photographing unit may alternatively be determined in another manner.

In some embodiments, the second height of the scanned subject may be a pre-stored height. For example, the scanned subject may be an imaging phantom. The dimensions of the imaging phantom are known. The present application is not limited thereto. The second height of the scanned subject may alternatively be determined in another manner.

FIG. 5 is a schematic diagram of a first height, a second height, a fourth distance, an offset, and a third distance according to an embodiment of the present application. As shown in (a) and (b) in FIG. 5, a vertical projection point (an image center) of a photographing unit on a reference plane is D, and the first height of the photographing unit from the reference plane is H. The second height of the scanned subject is h. Using a center point C of the scanned subject as an example, from the position of the photographing unit, the projection of the center point Con the reference plane is a point A, and the vertical projection point of the center point C on the reference plane is B. The position of the point A is the first position, a distance L between the point A and the point D is the fourth distance, and a distance between the point A and the point B is the offset. The reference object is located at a point E, that is, the position of the point E is the second position, and the distance between the point A and the point E is the third distance.

As shown in (a) and (b) in FIG. 5, an offset AB is the ratio of the product of the second height h and the fourth distance L to the first height H, that is, AB=h*L/H.

In some embodiments, in step 402, when the third distance is corrected according to the offset, a specific correction manner may be determined according to a positional relationship among the first position of the at least one point of the scanned subject in the first image, the second position of the reference object in the first image, and the image center of the first image.

For example, as shown in (a) in FIG. 5, when the first position A and the reference object E are located on the same side of the image center D, the first distance BE is a sum of the third distance AE and the offset AB.

For another example, as shown in (b) in FIG. 5, when the first position A and the reference object E are located on different sides of the image center D, the first distance BE is a difference between the third distance AE and the offset AB.

In some embodiments, the reference object may be various components of the medical imaging system, or may be a component outside the medical imaging system. For example, the reference object may be a component of which a relative position to the preset position is fixed. In this way, the scanned subject can be reliably moved to the preset position.

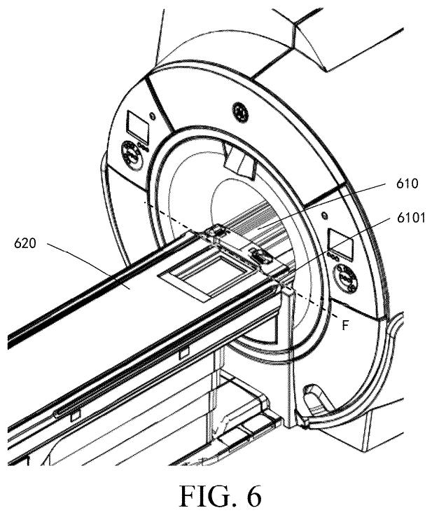

For example, the preset position may be a position of a scanning center of the medical imaging system, that is, an origin of a medical imaging coordinate system. The reference object may be a component of which a distance in the first direction in which the scanned subject is moved is fixed with respect to the scanning center. FIG. 6 is a schematic diagram of a partial structure of a medical imaging system according to an embodiment of the present application. As shown in FIG. 6, the reference object may be an edge 6101 of a bridge 610 of the medical imaging system. The bridge 610 is located in a magnet aperture of the medical imaging system and supported on a magnet in a front-rear direction, to support a table plate of an examination table 620 to move back and forth in the magnet. The edge 6101 of the bridge 610 is located at a position indicated by a dashed-dotted line F in FIG. 6. Because a distance between the edge 6101 of the bridge 610 and the scanning center is fixed, and the edge 6101 of the bridge 610 can be easily recognized through image recognition, the edge 6101 of the bridge 610 is used as a reference object, so that the position of the reference object can be reliably determined in the first image, and the accuracy of the first distance can be ensured.

The present application is not limited thereto. The reference object may alternatively be an identifiable position or an identifiable marker on a magnet housing. The identifiable position or the identifiable marker may be a built-in component of the medical imaging system or may be a position or a component additionally provided on the magnet housing. Alternatively, the reference object may alternatively be another identifiable object which is fixed relative to the magnet.

In some embodiments, the reference plane may be a plane with any height. For example, the reference plane may be a surface of an examination table of the medical imaging system for placing the scanned subject.

In some embodiments, the scanned subject may be various components for calibration of the medical imaging system. For example, the scanned subject may include an imaging phantom and the like. Because information of the scanned subject is known, the scanned subject is moved to the preset position, medical imaging (for example, pre-scanning) is performed on the scanned subject at the preset position, and a parameter of the medical imaging system can be calibrated according to a medical imaging result of the scanned subject and the known information of the scanned subject. For a specific calibration method, reference may be made to the related art. This does not be further explained here.

In some embodiments, there may be one or a plurality of scanned subjects. When there are a plurality of scanned subjects, for example, when the scanned subject includes a first scanned subject and a second scanned subject, the calibration method may further include: obtaining the first image including the reference object, the first scanned subject, and the second scanned subject, and determining the first distance according to the first image. The first distance includes a distance from at least one point of the first scanned subject to the reference object. The calibration method also includes moving the first scanned subject to the preset position according to the first distance and the second distance from the reference object to the preset position and calibrating a first parameter of the medical imaging system by using the first scanned subject at the preset position. The calibration method further includes determining a distance from the second scanned subject to the first scanned subject, that is, a distance between the first scanned subject and the second scanned subject, according to the first image; moving the second scanned subject to the preset position according to the distance from the second scanned subject to the first scanned subject; and calibrating a second parameter of the medical imaging system by using the second scanned subject at the preset position.

In some embodiments, the first parameter and the second parameter may be the same or different parameters.

In some embodiments, the first scanned subject and the second scanned subject may be the same or different scanned subjects. For example, the first scanned subject may be an imaging phantom related to a head, and the second scanned subject may be an imaging phantom related to a torso.

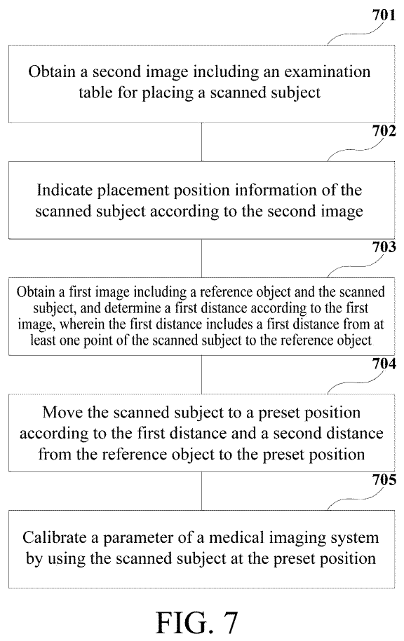

In some embodiments, to further simplify the calibration process, the placement position of the scanned subject may also be indicated to the operator. FIG. 7 is another schematic diagram of a calibration method for a medical imaging system according to an embodiment of the present application. As shown in FIG. 7, the method may include steps 701 to 705. At step 701 the method includes obtaining a second image including an examination table for placing a scanned subject and at step 702, the method includes indicating placement position information of the scanned subject according to the second image. Further, at step a first image including a reference object and the scanned subject is obtained, and a first distance is determined according to the first image, wherein the first distance includes a distance from at least one point of the scanned subject to the reference object. At step 704: the scanned subject is moved to a preset position according to the first distance and a second distance from the reference object to the preset position. At step 705 the method includes calibrating a parameter of the medical imaging system by using the scanned subject at the preset position.

According to the foregoing method, before the scanned subject is placed, the second image including at least the examination table is obtained, and the placement position of the scanned subject on the examination table is determined according to the second image, so that a user can place the scanned subject according to the indicated placement position information, thereby simplifying the calibration process.

In some embodiments, in step 702, the placement position information may be indicated in various manners. For example, the placement position information is displayed on a display, or is indicated by voice, vibration, or the like. This is not specifically limited in the present application.

In some embodiments, step 703 to step 705 may be the same as step 201 to step 203, and the content is incorporated herein and does not be further described.

The calibration process is exemplarily described below by using one scanned subject and two scanned subjects as examples.

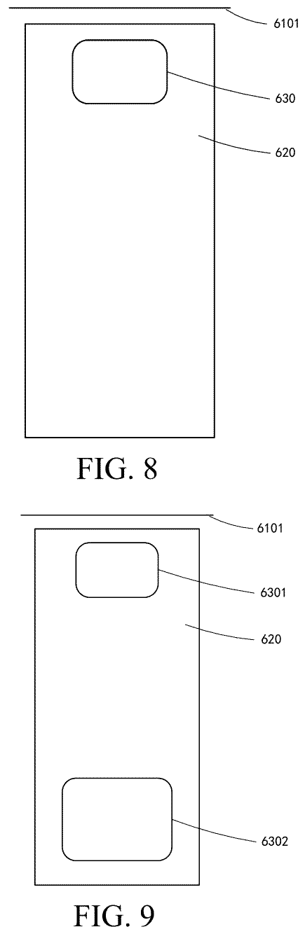

FIG. 8 is a schematic diagram of a scanned subject, a reference object, and an examination table. As shown in FIG. 8, there is one scanned subject 630, and the calibration process includes: obtaining a second image including the examination table 620; and indicating placement position information of the scanned subject 630 according to the second image. The calibration process also includes obtaining a first image including the reference object (the edge 6101 of the bridge 610) and the scanned subject 630 after the scanned subject 630 is placed on the examination table 620. Further, in the calibration process, a first distance from a center point of the scanned subject 630 to the reference object (the edge 6101 of the bridge 610) is determined according to the first image. Moreover, the examination table 620 is controlled according to the first distance and a second distance from the reference object (the edge 6101 of the bridge 610), to move the scanned subject 630 to the preset position to the preset position of a scanning center. Finally, a parameter of a medical imaging system is calibrated by using the scanned subject 630 at the preset position.

FIG. 9 is another schematic diagram of a scanned subject, a reference object, and an examination table. As shown in FIG. 9, there are two scanned subjects: a first scanned subject 6301 and a second scanned subject 6302, and the calibration process includes: obtaining a second image including the examination table 620 and indicating placement position information of the first scanned subject 6301 and the second scanned subject 6302 according to the second image. Further, the calibration process includes obtaining a first image including the reference object (the edge 6101 of the bridge 610), the first scanned subject 6301, and the second scanned subject 6302 after the first scanned subject 6301 and the second scanned subject 6302 are placed on the examination table 620. A first distance is determined according to the first image, where the first distance includes a distance from a center point of the first scanned subject 6301 to the reference object (the edge 6101 of the bridge 610). The process also includes controlling the examination table 620, according to the distance from the center point of the first scanned subject 6301 to the reference object (the edge 6101 of the bridge 610) and a second distance from the reference object (the edge 6101 of the bridge 610) to a preset position of a scanning center, to move the first scanned subject 6301 to the preset position. A first parameter of a medical imaging system is calibrated by using the first scanned subject 6301 at the preset position.

The process also includes determining a distance between the center point of the first scanned subject 6301 and a center point of the second scanned subject 6302 according to the first image. The examination table 620 is then controlled according to a distance from the center point of the second scanned subject 6302 to the center point of the first scanned subject 6301, to move the second scanned subject 6302 to the preset position. Finally, a second parameter of the medical imaging system is calibrated by using the second scanned subject 6302 at the preset position.

The method for calibrating the parameter of the medical imaging system by using the scanned subject is exemplarily described above. The present application is not limited thereto. The calibration of the medical imaging system may also include calibration between a medical imaging coordinate system and a camera coordinate system.

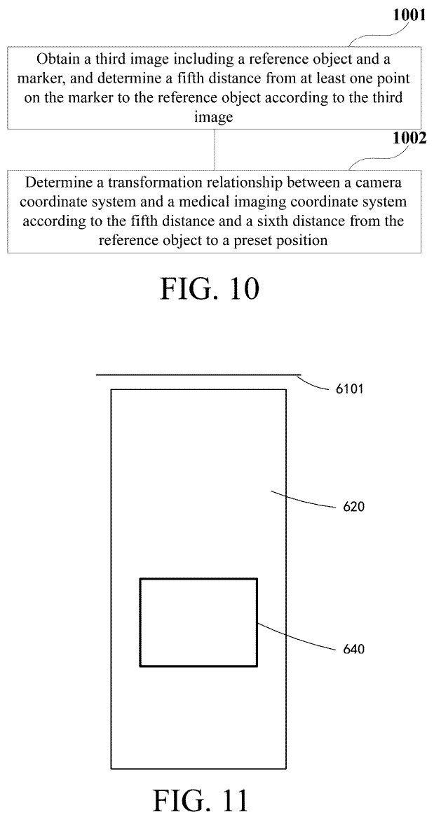

FIG. 10 is another schematic diagram of a calibration method for a medical imaging system according to an embodiment of the present application. As shown in FIG. 10, the method may include: at step 1001: obtaining a third image including a reference object and a marker, and determining a fifth distance from at least one point on the marker to the reference object according to the third image. The method also includes at step 1002: determining a transformation relationship between the camera coordinate system and the medical imaging coordinate system according to the fifth distance and a sixth distance from the reference object to a preset position.

According to the foregoing method, the third image including the reference object and the marker is obtained, the fifth distance from the at least one point on the marker to the reference object is determined according to the third image, and the transformation relationship between the camera coordinate system and the medical imaging coordinate system of the medical imaging system is determined according to the fifth distance and the sixth distance from the reference object to the preset position. In this way, the transformation relationship between the two coordinate systems is determined in a simple manner.

In some embodiments, there may be various transformation relationships between the camera coordinate system (Xc, Yc, Zc) of the medical imaging coordinate system (I, J, K), for example, affine transformation, which transforms a point (or position) in the camera coordinate system into a point (or position) in the medical imaging coordinate system by means of rotation and/or scaling and/or translation.

In some embodiments, the transformation relationship between the camera coordinate system (Xc, Yc, Zc) and the medical imaging coordinate system (I, J, K) may be used to perform position alignment on the two coordinate systems, and the transformation relationship may be represented by means of a transformation matrix.

In some embodiments, first coordinates of the at least one point on the marker in the camera coordinate system may be obtained according to the third image. A position relationship of the at least one point on the marker relative to the reference object may alternatively be obtained according to the third image, and second coordinates of the at least one point on the marker in the medical imaging coordinate system may be determined according to the position relationship and a position relationship between the reference object and the preset position (for example, the scanning center). Therefore, the transformation matrix between the two coordinate systems can be determined according to the first coordinates and the second coordinates.

In some embodiments, the marker may be a black and white checkerboard pattern. The present application is not limited thereto. The marker may alternatively be another pattern.

FIG. 11 is a schematic diagram of a marker, a reference object, and an examination table according to an embodiment of the present application. As shown in FIG. 11, the reference object (the edge 6101 of the bridge 610) and the marker 640 are placed on the examination table 620, and the calibration process includes: obtaining a third image including the reference object (the edge 6101 of the bridge 610) and the marker 640 and determining first coordinates of at least one point on the marker 640 in a camera coordinate system according to the third image. A fifth distance from the at least one point on the marker 640 to the reference object (the edge 6101 of the bridge 610) is then determined according to the third image. Second coordinates of the at least one point on the marker 640 in a medical imaging coordinate system are determined according to the fifth distance and a sixth distance from the reference object 640 to a preset position. Further, the process includes determining a transformation relationship between the camera coordinate system and the medical imaging coordinate system according to the first coordinates and the second coordinates.

In some embodiments, step 1001 and step 1002 are optional steps. In a case that step 1001 and step 1002 are performed, these steps may be performed before step 201 to step 203, or may be performed in parallel with step 201 to step 203, or may be performed after step 201 to step 203.

In some embodiments, calibrated parameters may be various parameters of the medical imaging system. For example, the calibrated parameters may include the position of the scanning center of the medical imaging system. Because the influence of factors such as manufacturing and assembly, the actual position of the scanning center of the medical imaging system may be different from the preset position. The preset position may be an estimated/theoretical position of the scanning center. The second distance between the reference object and the scanning center may be a distance determined according to the preset position. After the parameters of the medical imaging system are calibrated, the actual position of the scanning center of the medical imaging system may be determined.

If step 1001 and step 1002 are performed after step 201 to step 203, the sixth distance in step 1002 may be a distance obtained by correcting the second distance according to a calibration result of the medical imaging system. Therefore, the accuracy of the transformation relationship between the two coordinate systems can be further improved. The present application is not limited thereto. The sixth distance may alternatively be the second distance.

In some embodiments, the distance in the embodiments of the present application may be represented by the number of pixels of an image. The present application is not limited thereto. The distance in the embodiments of the present application may alternatively be represented by using a physical length. In this case, the distance may be determined according to the number of pixels and the physical length corresponding to each pixel.

The physical length corresponding to each pixel may be determined according to the number of pixels of a preset component in the image and the actual physical length of the preset component. For example, the preset component may be an examination table, the examination table is identified in the image by a template matching algorithm or the like, and the number of pixels of the examination table is determined, for example, 524 pixels. The actual length of the examination table is known, for example, 1134 mm, and the length corresponding to each pixel is R=1134/524=2.1641 mm/pixel. The present application is not limited thereto. The length corresponding to each pixel may alternatively be determined in another manner, or the distance may be represented in another manner.

In some embodiments, when the position of the scanned subject (for example, the first position) or the position of the reference object (for example, the second position) is determined according to the image, the positions of a plurality of points on the scanned subject or the reference object may be determined first, and the average value of the plurality of positions may be used as the position of the scanned subject or the reference object. The present application is not limited thereto. The position of the scanned subject or the reference object may alternatively be determined in another manner.

It should be noted that the above figures merely schematically illustrate the embodiments of the present application, but the present application is not limited thereto. For example, the order of execution between operations may be appropriately adjusted. In addition, some other operations may be added or some operations may be omitted. Those skilled in the art can make appropriate variations according to the above content, rather than being limited by the disclosure of the foregoing accompanying drawings.

The foregoing embodiments merely provide illustrative descriptions of the embodiments of the present application. However, the present application is not limited thereto, and appropriate variations may be made on the basis of the foregoing embodiments. For example, each of the embodiments described above may be used independently, or one or more among the foregoing embodiments may be combined.

According to the foregoing embodiment, the first image including the reference object and the scanned subject is obtained, and the first distance from the at least one point of the scanned subject to the reference object is determined according to the first image; the scanned subject is moved to the preset position according to the first distance and the second distance from the reference object to the preset position; and the parameter of the medical imaging system is calibrated by using the scanned subject at the preset position. In this way, the accuracy of a calibration result of the medical imaging system can be improved, which is beneficial to simplify the calibration process and reduce the costs of the medical imaging system.

An embodiment of the present application further provides a calibration apparatus for a medical imaging system. The content that is the same as the foregoing embodiments does not be repeated.

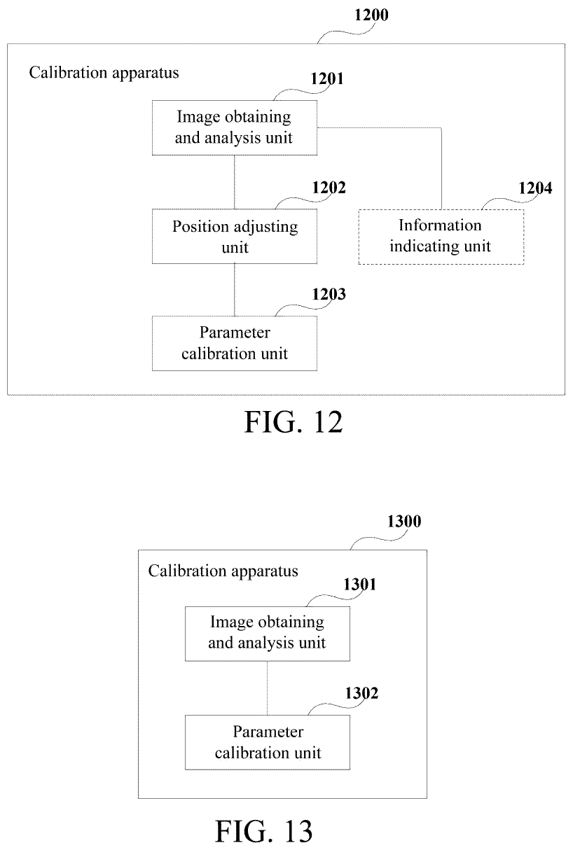

FIG. 12 is a schematic diagram of a calibration apparatus for a medical imaging system according to an embodiment of the present application. As shown in FIG. 12, the calibration apparatus 1200 for a medical imaging system includes: an image obtaining and analysis unit 1201, configured to obtain a first image including a reference object and a scanned subject, and determine a first distance according to the first image, wherein the first distance includes a distance from at least one point of the scanned subject to the reference object.

The calibration apparatus also includes a position adjusting unit 1202, configured to move the scanned subject to a preset position according to the first distance and a second distance from the reference object to the preset position; and a parameter calibration unit 1203, configured to calibrate a parameter of the medical imaging system by using the scanned subject at the preset position.

In some embodiments, the scanned subject is moved to the preset position in a first direction, and the first distance includes a distance in the first direction from at least one point of the scanned subject to the reference object.

In some embodiments, the image obtaining and analysis unit 1201 determines a third distance between a first position of the at least one point of the scanned subject in the first image and a second position of the reference object in the first image; and corrects the third distance according to a first height of a photographing unit that captures the first image and a second height of the scanned subject to obtain the first distance.

In some embodiments, the image obtaining and analysis unit 1201 determines an offset according to the first height, the second height, and a fourth distance between the first position and an image center of the first image; and corrects the third distance according to the offset to obtain the first distance.

In some embodiments, the offset is the ratio of the product of the second height and the fourth distance to the first height.

In some embodiments, when the first position and the reference object are located on the same side of the image center, the first distance is a sum of the third distance and the offset. Alternatively, when the first position and the reference object are located on different sides of the image center, the first distance is a difference between the third distance and the offset.

In some embodiments, the first height and the second height are heights with respect to a same reference plane.

In some embodiments, the reference object is at the same height as the reference plane, or a height difference between the reference object and the reference plane is less than a preset value.

In some embodiments, the reference object is an edge of a bridge of the medical imaging system.

In some embodiments, the reference plane is a surface of an examination table of the medical imaging system for placing the scanned subject.

In some embodiments, the image obtaining and analysis unit 1201 further obtains a second image including the examination table for placing the scanned subject. The apparatus 1200 further includes an information indicating unit 1204. The information indicating unit 1204 indicates placement position information of the scanned subject according to the second image.

In some embodiments, when the scanned subject includes a first scanned subject and a second scanned subject, the image obtaining and analysis unit 1201 obtains the first image including the reference object, the first scanned subject, and the second scanned subject, and determines the first distance according to the first image, wherein the first distance includes a distance from at least one point of the first scanned subject to the reference object. The position adjusting unit 1202 moves the first scanned subject to a preset position according to the first distance and a second distance from the reference object to the preset position. The parameter calibration unit 1203 calibrates a first parameter of the medical imaging system by using the first scanned subject at the preset position. The image obtaining and analysis unit 1201 determines a distance between the first scanned subject and the second scanned subject according to the first image. The position adjusting unit 1202 moves the second scanned subject to the preset position according to the distance between the first scanned subject and the second scanned subject. The parameter calibration unit 1203 calibrates a second parameter of the medical imaging system by using the second scanned subject at the preset position.

In some embodiments, the image obtaining and analysis unit 1201 obtains a third image including the reference object and a marker, and determines a fifth distance from at least one point on the marker to the reference object according to the third image. The parameter calibration unit 1203 determines a transformation relationship between a camera coordinate system and a medical imaging coordinate system according to the fifth distance and a sixth distance from the reference object to the preset position.

In some embodiments, the sixth distance is the second distance, or the sixth distance is a distance obtained by correcting the second distance according to a calibration result of the medical imaging system.

It is worth noting that only the components or modules related to the present application have been described above, but the present application is not limited thereto. The calibration apparatus for a medical imaging system may further include other components or modules, or omit some components or modules (for example, a component or module corresponding to a dashed-line box in the figure). For the specific content of these components or modules, reference may be made to the related art.

In addition, for simplicity, the above figures only exemplarily illustrate connection relationships or signal directions between various components or modules, but it should be clear to those skilled in the art that various related technologies such as bus connection may be used. The various components or modules may be implemented by means of hardware facilities such as a processor, a memory, a transmitter and a receiver. The implementation of the present application is not limited thereto.

According to the foregoing embodiment, the first image including the reference object and the scanned subject is obtained, and the first distance from the at least one point of the scanned subject to the reference object is determined according to the first image; the scanned subject is moved to the preset position according to the first distance and the second distance from the reference object to the preset position; and the parameter of the medical imaging system is calibrated by using the scanned subject at the preset position. In this way, the accuracy of the calibration result of the medical imaging system can be improved, which is beneficial to simplify the calibration process and reduce the costs of the medical imaging system.

An embodiment of the present application provides a calibration method for a medical imaging system. As shown in FIG. 10, the calibration method for a medical imaging system includes: obtaining a third image including a reference object and a marker, and determining a fifth distance from at least one point on the marker to the reference object according to the third image; and determining a transformation relationship between the camera coordinate system and the medical imaging coordinate system according to the fifth distance and a sixth distance from the reference object to a preset position.

In other words, the method shown in FIG. 10 may alternatively be performed independently of the method shown in FIG. 2, and the content that is the same as the foregoing embodiments does not be repeated.

It should be noted that the above figures merely schematically illustrate the embodiments of the present application, but the present application is not limited thereto. For example, the order of execution between operations may be appropriately adjusted. In addition, some other operations may be added, or some operations may be omitted (for example, operations or steps corresponding to dashed boxes in the figures). Those skilled in the art can make appropriate variations according to the above content, rather than being limited by the disclosure of the foregoing accompanying drawings.

The foregoing embodiments merely provide illustrative descriptions of the embodiments of the present application. However, the present application is not limited thereto, and appropriate variations may be made on the basis of the foregoing embodiments. For example, each of the embodiments described above may be used independently, or one or more among the foregoing embodiments may be combined.

According to the foregoing embodiment, the third image including the reference object and the marker is obtained, the fifth distance from at least one point on the marker to the reference object is determined according to the third image, and the transformation relationship between the camera coordinate system and the medical imaging coordinate system of the medical imaging system is determined according to the fifth distance and the sixth distance from the reference object to the preset position. In this way, the transformation relationship between the two coordinate systems is determined in a simple manner.

An embodiment of the present application further provides a calibration apparatus for a medical imaging system. The content that is the same as the foregoing embodiments does not be repeated.

FIG. 13 is another schematic diagram of a calibration apparatus for a medical imaging system according to an embodiment of the present application. As shown in FIG. 13, the calibration apparatus 1300 for a medical imaging system includes: an image obtaining and analysis unit 1301, configured to obtain a third image including a reference object and a marker, and determine a fifth distance from at least one point on the marker to the reference object according to the third image. The calibration apparatus 1300 also includes a parameter correction unit 1302, configured to determine a transformation relationship between a camera coordinate system and a medical imaging coordinate system according to the fifth distance and a sixth distance from the reference object to a preset position.

It is worth noting that only the components or modules related to the present application have been described above, but the present application is not limited thereto. The calibration apparatus for a medical imaging system may further include other components or modules, or omit some components or modules (for example, a component or module corresponding to a dashed-line box in the figure). For the specific content of these components or modules, reference may be made to the related art.

In addition, for simplicity, the above figures only exemplarily illustrate connection relationships or signal directions between various components or modules, but it should be clear to those skilled in the art that various related technologies such as bus connection may be used. The various components or modules may be implemented by means of hardware facilities such as a processor, a memory, a transmitter and a receiver. The implementation of the present application is not limited thereto.

According to the foregoing embodiment, the third image including the reference object and the marker is obtained, the fifth distance from at least one point on the marker to the reference object is determined according to the third image, and the transformation relationship between the camera coordinate system and the medical imaging coordinate system of the medical imaging system is determined according to the fifth distance and the sixth distance from the reference object to the preset position. In this way, the transformation relationship between the two coordinate systems is determined in a simple manner.

Embodiments of the present application further provide a medical imaging system. The configuration of the medical imaging system is as shown in FIG. 1, and repeated portions will not be described again.

In some embodiments, the medical imaging system may include the foregoing calibration apparatus 1200. The calibration apparatus 1200 includes: an image obtaining and analysis unit 1201, configured to obtain a first image including a reference object and a scanned subject, and determine a first distance according to the first image, wherein the first distance includes a distance from at least one point of the scanned subject to the reference object.

The calibration apparatus 1200 also includes a position adjusting unit 1202, configured to move the scanned subject to a preset position according to the first distance and a second distance from the reference object to the preset position; and a parameter calibration unit 1203, configured to calibrate a parameter of the medical imaging system by using the scanned subject at the preset position.

In some embodiments, the image obtaining and analysis unit 1201 obtains a third image including the reference object and a marker, and determines a fifth distance from at least one point on the marker to the reference object according to the third image. The parameter calibration unit 1203 determines a transformation relationship between a camera coordinate system and a medical imaging coordinate system according to the fifth distance and a sixth distance from the reference object to the preset position.

In some embodiments, the sixth distance is the second distance, or the sixth distance is a distance obtained by correcting the second distance according to a calibration result of the medical imaging system.

In some embodiments, the image capture apparatus 180 shown in FIG. 1 may capture an image (for example, at least one of the first image, the second image, or the third image as described above) in response to an instruction of the image obtaining and analysis unit 1201. The image obtaining and analysis unit 1201 may include the controller 130 shown in FIG. 1 or another processor. The controller 130 or another processor obtains the captured image from the image capture apparatus 180 or a memory, and obtains a distance relationship between subjects in the image based on the image.

In some embodiments, the position adjusting unit 1202 may be the controller 130 or another processor or the patient positioning system 147 shown in FIG. 1, and communicates with an examination table to control the examination table to move, and so on.

In some embodiments, the parameter calibration unit 1203 may be the calibration module 134 shown in FIG. 1 or another processor.

In some embodiments, the medical imaging system may include the foregoing calibration apparatus 1300. The calibration apparatus 1300 includes: an image obtaining and analysis unit 1301, configured to obtain a third image including a reference object and a marker, and determine a fifth distance from at least one point on the marker to the reference object according to the third image. The calibration apparatus 1300 also includes a parameter correction unit 1302, configured to determine a transformation relationship between a camera coordinate system and a medical imaging coordinate system according to the fifth distance and a sixth distance from the reference object to a preset position.

In some embodiments, the image obtaining and analysis unit 1201 may include the controller 130 shown in FIG. 1 or another processor. The controller 130 or another processor obtains the captured image from the image capture apparatus 180 or a memory, and obtains a distance relationship between subjects in the image based on the image.

In some embodiments, the parameter correction unit 1302 may be the calibration module 134 shown in FIG. 1 or another processor.

In some embodiments, the medical imaging system includes a controller and an examination table. The controller (for example, the controller 130) may perform the foregoing calibration method for a medical imaging system. The controller 130 may include a computer processor and a storage medium. The storage medium records a predetermined data processing program to be executed by the computer processor. For example, the storage medium may store a program configured to implement scanning processing (for example, including waveform design/conversion, and the like), image reconstruction, medical imaging, and the like. For example, the storage medium may store a program configured to implement a calibration method for a medical imaging system according to an embodiment of the present invention. Specific implementations of the calibration method for a medical imaging system are described above, and do not be described again here. The described storage medium may include, for example, a ROM, a floppy disk, a hard disk, an optical disk, a magneto-optical disk, a CD-ROM, or a non-volatile memory card.

The present application is not limited thereto. The foregoing calibration method for a medical imaging system may alternatively be performed in another processor in the MRI system 100, or by a cloud processor.

The examination table is configured to place a scanned subject, and move the scanned subject to the preset position according to control of the controller.

According to the foregoing embodiment, the first image including the reference object and the scanned subject is obtained, and the first distance from the at least one point of the scanned subject to the reference object is determined according to the first image; the scanned subject is moved to the preset position according to the first distance and the second distance from the reference object to the preset position; and the parameter of the medical imaging system is calibrated by using the scanned subject at the preset position. In this way, the accuracy of the calibration result of the medical imaging system can be improved, which is beneficial to simplify the calibration process and reduce the costs of the medical imaging system.

An embodiment of the present application further provides a computer-readable program. When the program is executed in a medical imaging system, the program causes a computer to perform, in the medical imaging system, the calibration method for a medical imaging system according to the foregoing embodiments.

An embodiment of the present application further provides a storage medium having a computer-readable program stored therein. The computer-readable program causes a computer to perform, in a medical imaging system, the calibration method for a medical imaging system according to the foregoing embodiments.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A calibration method for a medical imaging system, characterized by comprising:
obtaining a first image comprising a reference object and a scanned subject, and determining a first distance according to the first image, wherein the first distance comprises a distance from at least one point of the scanned subject to the reference object;
moving the scanned subject to a preset position according to the first distance and a second distance from the reference object to the preset position; and
calibrating a parameter of the medical imaging system by using the scanned subject at the preset position.

2. The method according to claim 1, wherein
the scanned subject is moved to the preset position in a first direction, and the first distance comprises a distance in the first direction from the at least one point of the scanned subject to the reference object.

3. The method according to claim 1, wherein the determining the first distance according to the first image comprises:
determining a third distance between a first position of the at least one point of the scanned subject in the first image and a second position of the reference object in the first image; and
correcting the third distance according to a first height of a photographing unit that captures the first image and a second height of the scanned subject to obtain the first distance.

4. The method according to claim 3, wherein the correcting the third distance according to the first height of the photographing unit that captures the first image and the second height of the scanned subject to obtain the first distance comprises:
determining an offset according to the first height, the second height, and a fourth distance between the first position and an image center of the first image; and
correcting the third distance according to the offset to obtain the first distance.

5. The method according to claim 4, wherein
the offset is the ratio of the product of the second height and the fourth distance to the first height.

6. The method according to claim 4, wherein
when the first position and the reference object are located on the same side of the image center, the first distance is a sum of the third distance and the offset; or
when the first position and the reference object are located on different sides of the image center, the first distance is a difference between the third distance and the offset.

7. The method according to claim 3, wherein
the first height and the second height are heights with respect to a same reference plane.

8. The method according to claim 7, wherein
the reference object is at the same height as the reference plane; or
the reference object is an edge of a bridge of the medical imaging system; or
the reference plane is a surface of an examination table of the medical imaging system for placing the scanned subject.

9. The method according to claim 1, further comprising:
obtaining a second image comprising an examination table for placing the scanned subject; and
indicating placement position information of the scanned subject according to the second image.

10. The method according to claim 1, wherein
when the scanned subject comprises a first scanned subject and a second scanned subject, the method further comprises:
obtaining the first image comprising the reference object, the first scanned subject, and the second scanned subject, and determining the first distance according to the first image, wherein the first distance comprises a distance from at least one point of the first scanned subject to the reference object;
moving the first scanned subject to the preset position according to the first distance and the second distance from the reference object to the preset position;
calibrating a first parameter of the medical imaging system by using the first scanned subject at the preset position;
determining a distance between the first scanned subject and the second scanned subject according to the first image;
moving the second scanned subject to the preset position according to the distance between the first scanned subject and the second scanned subject; and
calibrating a second parameter of the medical imaging system by using the second scanned subject at the preset position.

11. The method according to claim 1, further comprising:
obtaining a third image comprising the reference object and a marker, and determining a fifth distance from at least one point on the marker to the reference object according to the third image; and
determining a transformation relationship between a camera coordinate system and a medical imaging coordinate system according to the fifth distance and a sixth distance from the reference object to the preset position.

12. The method according to claim 11, wherein
the sixth distance is the second distance, or the sixth distance is a distance obtained by correcting the second distance according to a calibration result of the medical imaging system.

13. A medical imaging system, characterized by comprising:
a controller, configured to perform the calibration method for a medical imaging system according to claim 1; and
an examination table, configured for placing a scanned subject and moving the scanned subject to a preset position.

14. The system according to claim 13, wherein
the controller is further configured to:
obtain a third image comprising the reference object and a marker, and determine a fifth distance from at least one point on the marker to the reference object according to the third image; and
determine a transformation relationship between a camera coordinate system and a medical imaging coordinate system according to the fifth distance and a sixth distance from the reference object to the preset position, wherein the sixth distance is the second distance, or the sixth distance is a distance obtained by correcting the second distance according to a calibration result of the medical imaging system.
